(19) United States
(12) Patent Application Publication
(10) Pub. No.: US 2010/0268084 A1
(43) Pub. Date: Oct. 21, 2010

(54) ULTRASONIC DIAGNOSTIC APPARATUS

(76) Inventors: Takashi Osaka, Tokyo (JP); Akiko Tonomura, Tokyo (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/741,405

(22) PCT Filed: Oct. 24, 2008
(86) PCT No.: PCT/JP2008/069346

§ 371 Date: May 5, 2010

(30) Foreign Application Priority Data
Nov. 6, 2007 (JP) 2007-288766

(51) Int. Cl. A61B 8/00

(52) U.S. Cl. 600/443

(57) ABSTRACT

An ultrasonic diagnostic apparatus enabling accurate measurement of elasticity information is provided.

The ultrasonic diagnostic apparatus is characterized in comprising setting means configured to set a first movement reference point and a second movement reference point on a tomographic image and/or an elastic image, and measurement means configured to cause the first movement reference point and the second movement reference point to follow the pulsation of an object and to measure the length of a line segment formed by the first movement reference point and the second movement reference point, and causes display means to display the length of the line segment and elasticity information based on the strain and/or elastic modulus.

### TECHNICAL FIELD

The present invention is related to the ultrasonic diagnostic apparatus that displays tomographic images or elastic images which represents hardness or softness of biological tissues by transmitting/receiving ultrasonic waves to/from an imaging target in an object to be examined.

### BACKGROUND ART

Ultrasonic diagnostic apparatuses transmit an ultrasonic wave to an object to be examined using an ultrasonic probe and receive from the object the reflected echo signal of the ultrasonic wave in accordance with the constitution of the biological tissues, so as to construct and display, for example, a tomographic image.

In recent years, a technique has been disclosed that measures an ultrasonic reception signal by compressing the object by an ultrasonic probe using manual or mechanical method, acquiring the displacement in the respective regions of the biological tissues generated by the compression based on RF frame data of two ultrasonic reception signals having different measurement times, and generates an elastic image representing elasticity information of the biological tissues based on the displacement data. Also, the technique detects the movement of the biological tissue by electrocardiographic waveforms, reads out the RF frame data of movement of the biological tissue based on the reference time phase, and generates an elastic image (for example, Patent Document 1).

Patent Document 1: WO2006/132203

### DISCLOSURE OF THE INVENTION

### Problems to be Solved

In Patent Document 1, since the R-wave of electro-cardiographic waveform by the heart of an object is set as the reference point and the RF frame data in the time phase which is displaced for a predetermined time phase from the reference point is read out, there is a possibility that the displacement of the time phase for reading out the RF frame data is caused depending on the object. In such cases, it is impossible to control the compression, or to execute accurate measurement of elasticity information under a specific compression which repeats precipitous application of pressure and moderate reduction of pressure on a periodic basis.

The objective of the present invention is to execute measurement of elasticity information accurately.

### Means to Solve the Problem

In order to achieve the objective of the present invention, the ultrasonic diagnostic apparatus comprising:

an ultrasonic probe,

tomographic image constructing means configured to generate a tomographic image based on the RF frame data of a cross-sectional region of an object to be examined obtained by the ultrasonic probe;

elasticity information calculating means configured to obtain strain and/or elasticity modulus of tissues in the cross-section region based on the RF frame data;

elastic image constructing means configured to generate an elastic image in the cross-section region based on the strain and/or the elasticity modulus obtained by the elasticity information calculating means; and

display means configured to display the tomographic image or the elastic image,

characterized in further comprising:

setting means configured to set a first movement reference point and a second movement reference point on the tomogrpahic image or the elastic image; and

measuring means configured to cause the first movement reference point and the second movement reference point to follow the pulsation of the object and to measure the length of a line segment formed by the first movement reference point and the second movement reference point,

and causes display means to display the length of the line segment and elastic information based on the strain and/or the elasticity modulus.

The setting means sets a region of interest in the vicinity of the first movement reference point or the second movement reference point, and causes display means to display the elasticity information within the region of interest.

The elasticity information is the average strain value within the region interest. The elasticity information is the strain ratio between the two regions of interest.

The display means displays a time series graph based on the length of the line segment and/or the elasticity information. The display means displays a time gauge indicating the time phase on the time series graph.

The measurement means measures the length of the line segment or the elasticity information in a predetermined time zone.

The display means changes the display pattern of the elastic image based on the variation information of the length of the line segment. Also, the display means changes the display pattern of the time series graph based on the variation information of the length of the line segment.

Further, the setting means modifies the setting position of the region of interest in accordance with the pressure.

### EFFECT OF THE INVENTION

In accordance with the present invention, it is possible to execute accurate measurement of elasticity information.

### BRIEF DESCRIPTION OF THE DIAGRAMS

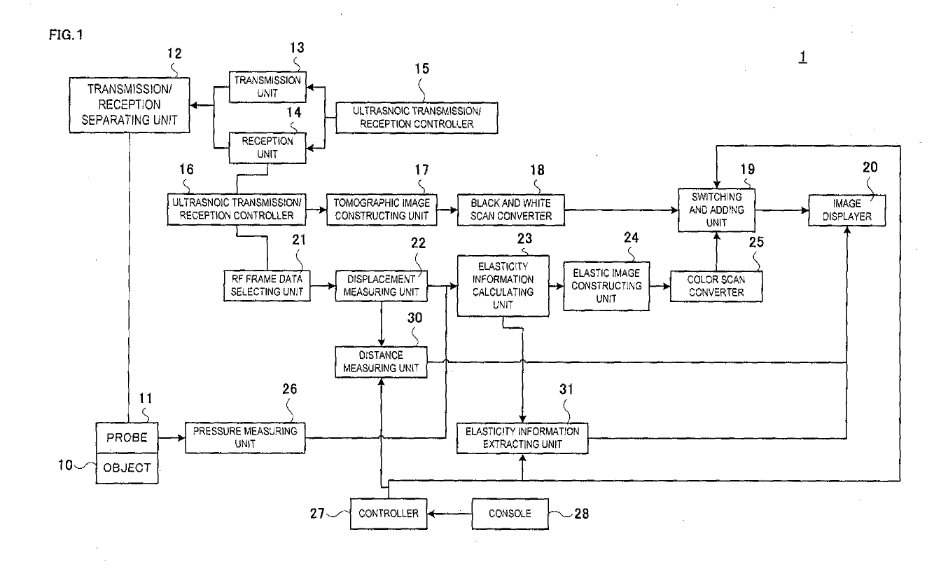

FIG. 1 is a configuration block diagram of the present invention.

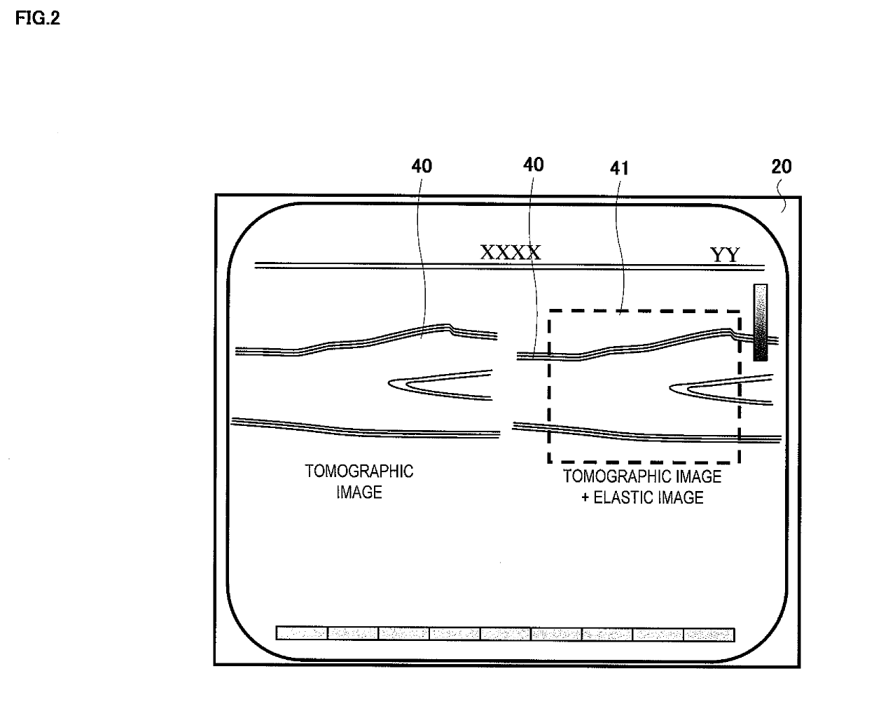

FIG. 2 is the display example of image display unit 20 related to the present invention.

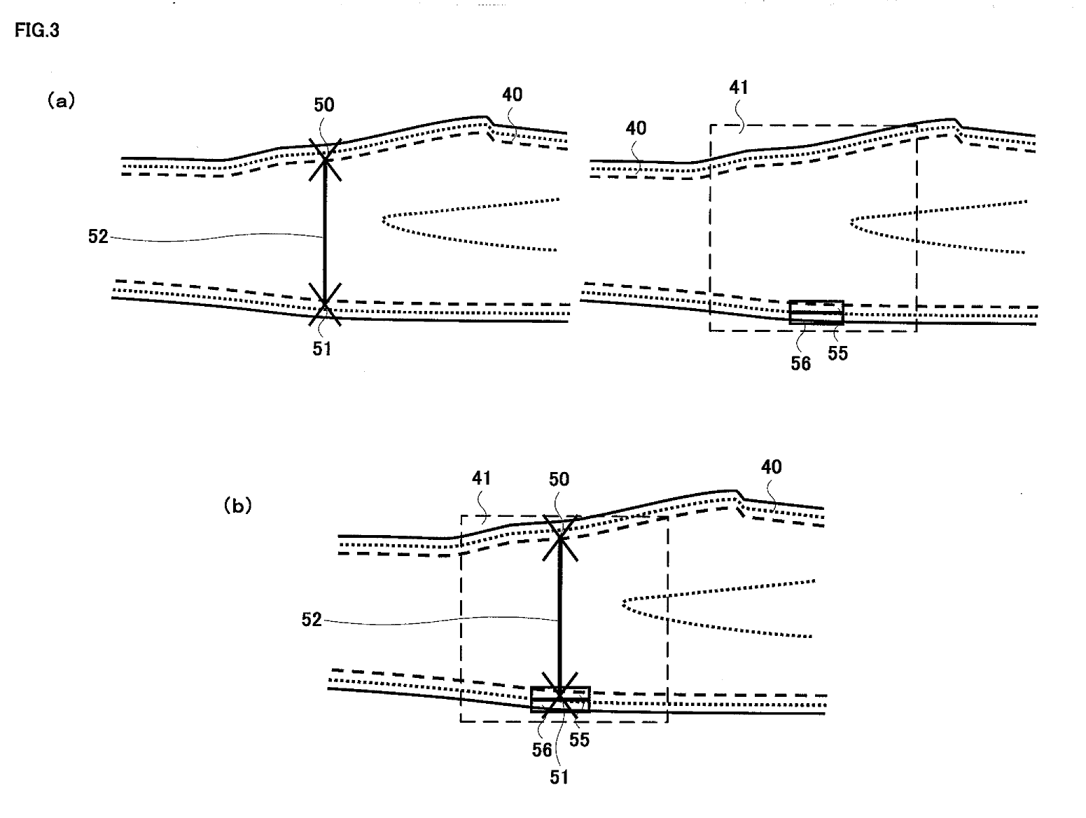

FIG. 3 shows the first embodiment of the present invention.

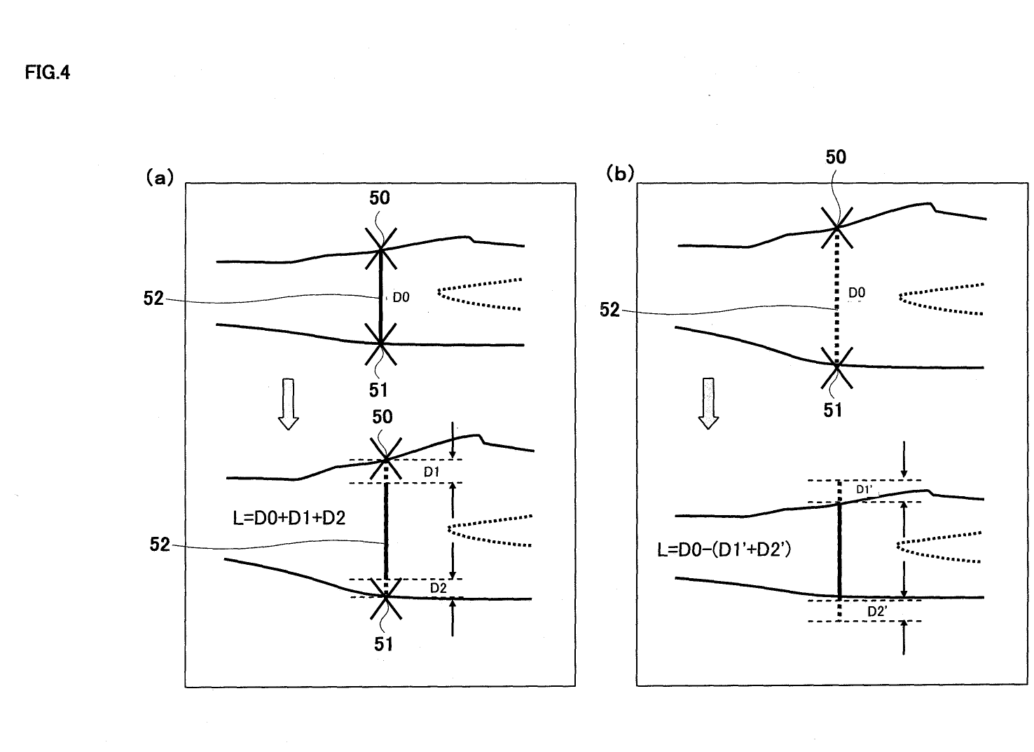

FIG. 4 shows the first embodiment of the present invention.

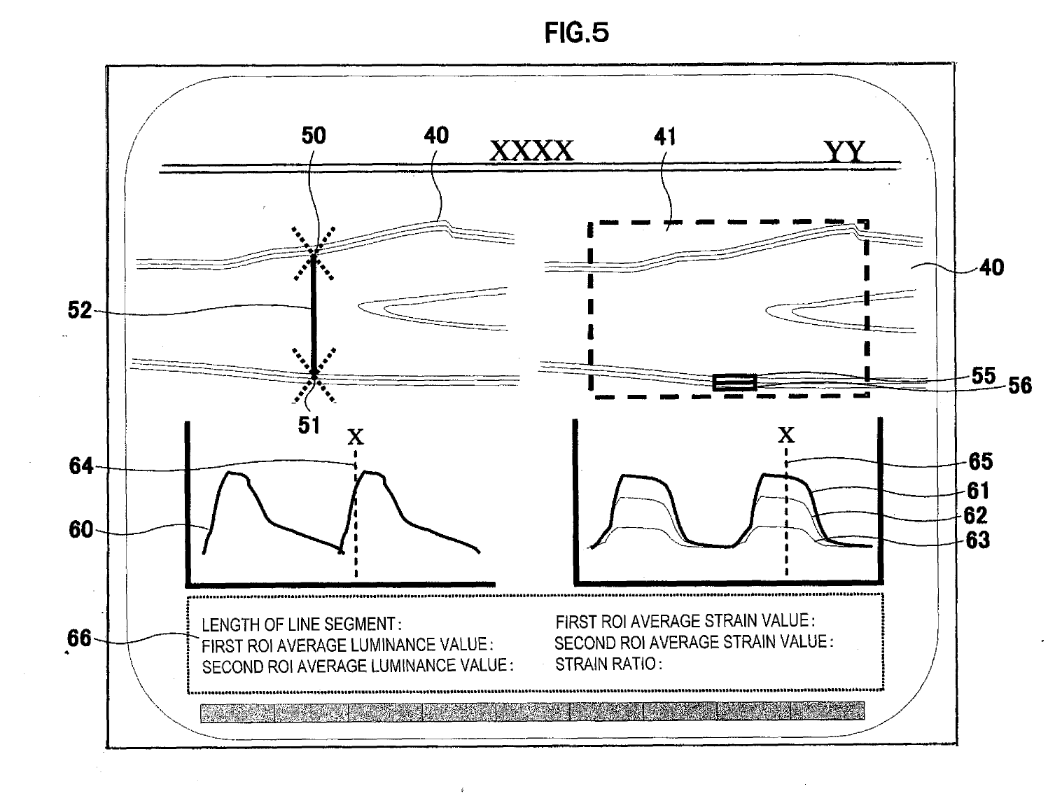

FIG. 5 shows the first embodiment and the fourth embodiment of the present invention.

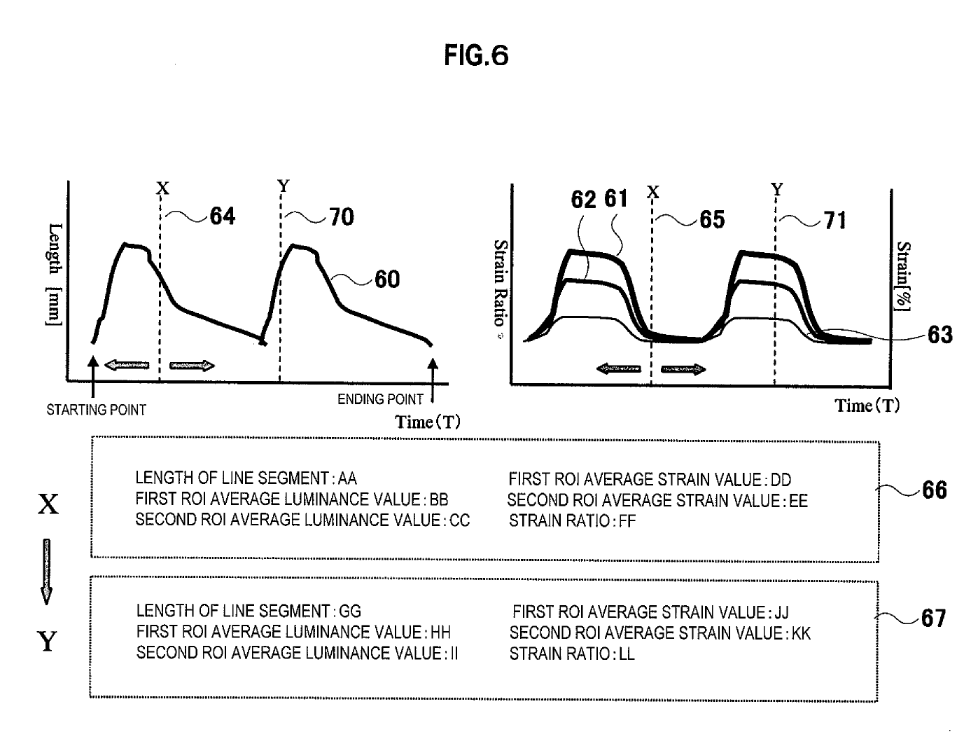

FIG. 6 shows the first embodiment, the second embodiment and the sixth embodiment of the present invention.

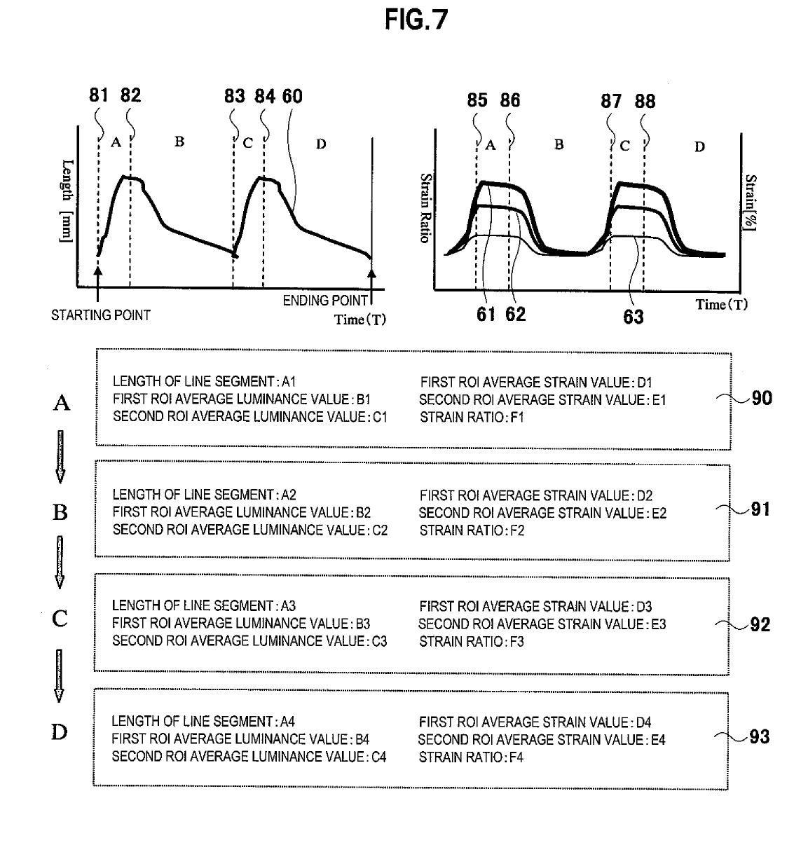

FIG. 7 shows the third embodiment and the fourth embodiment of the present invention.

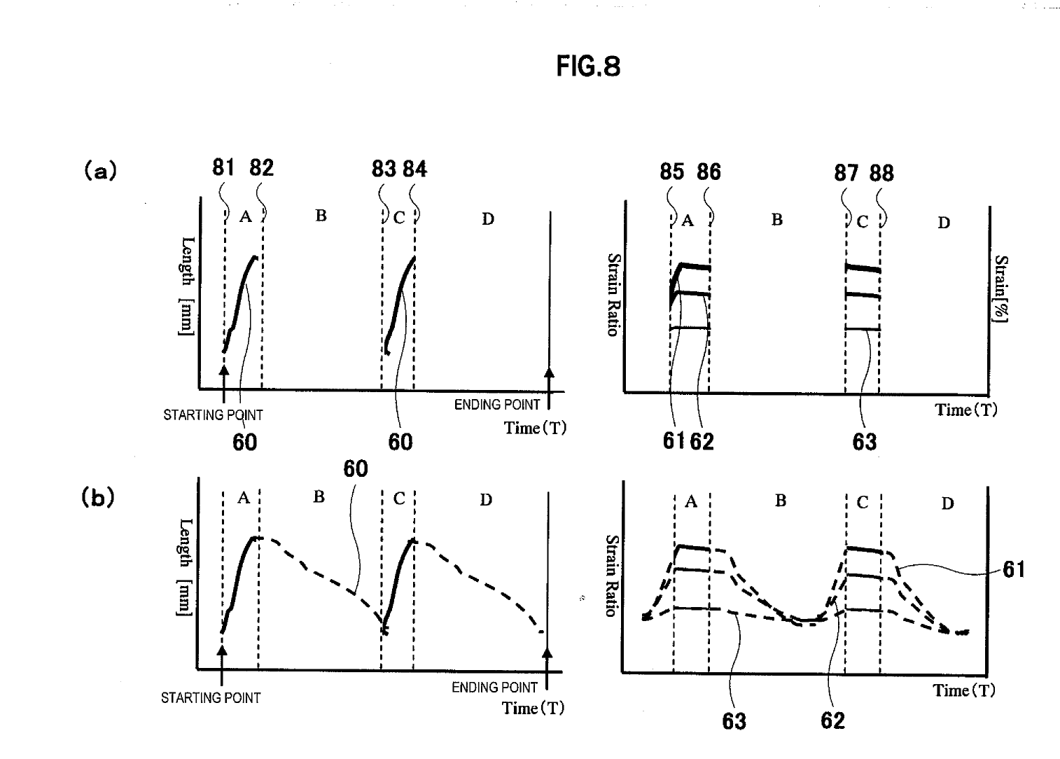

FIG. 8 shows the fifth embodiment of the present invention.

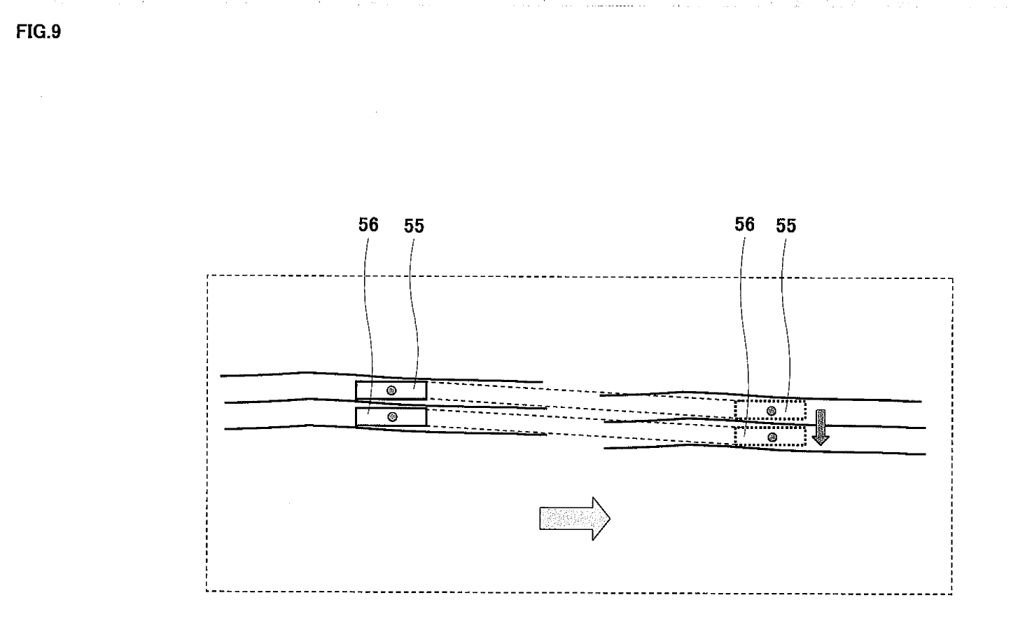

FIG. 9 shows the sixth embodiment of the present invention.

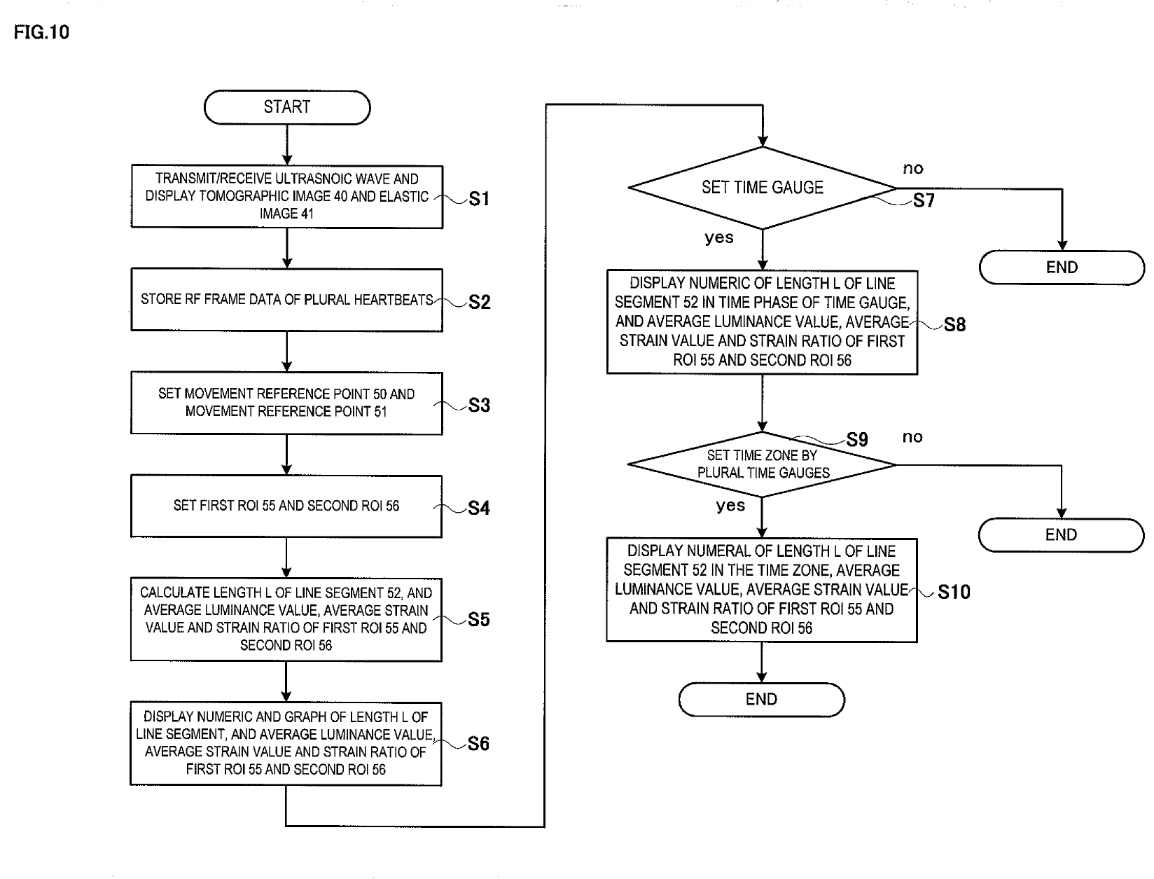

FIG. 10 shows an operation procedure of the present invention.

### DESCRIPTION OF THE REFERENCE NUMERALS

10: object, 11: probe, 12: transmission/reception separating unit, 13: transmission unit, 14: reception unit, 15: ultrasonic transmission/reception controller, 16: phasing and adding unit, 17: tomographic image constructing unit, 18: black and white scan converter, 19: switching and adding unit, 20: image display unit, 21: RF frame data selecting unit, 22: displacement measuring unit, 23: elasticity information calculating unit, 24: elastic image constructing unit, 25: color scan converter, 26: pressure measurement unit, 27: controller, 28: console, 30: distance measuring unit, 31: elasticity information extracting unit

### BEST MODE FOR CARRYING OUT THE INVENTION

Ultrasonic diagnostic apparatus 1 to which the present invention is applied will be described referring to FIG. 1. FIG. 1 is a block diagram showing components of ultrasonic diagnostic apparatus 1 to which the present invention is applied.

As shown in FIG. 1, ultrasonic diagnostic apparatus 1 comprises:

ultrasonic probe 11 configured to use by applying to object 10;

transmission unit 13 configured to repeatedly transmit ultrasonic waves to object 10 at time intervals via ultrasonic probe 11;

reception unit 14 configured to receive time-series reflected echo signals produced from object 10;

transmission/reception separating unit 12 configured to separate ultrasonic waves for transmission and reflected echoes;

ultrasonic transmission/reception controller 15 configured to control transmission unit 13 and reception unit 14; and

phasing and adding means 16 configured to phase and add the reflected echoes received in reception unit 14.

It also comprises:

tomographic image constructing unit 17 configured to construct a grayscale tomographic image, for example, black and white tomographic image of object 10 based on the RF frame data from phasing and adding unit 16; and

black and white scan converter 18 configured to convert the output signals from tomographic constructing unit 17 so as to match the output signals to display of image display unit 20.

Further, it comprises:

RF frame data selecting unit 21 configured to store the RF frame data outputted from phasing and adding unit 16, and to select at least two sets of RF frame data;

displacement measuring unit 22 configured to measure the displacement of biological tissues of object 10 from the selected RF frame data;

elasticity information calculating unit 23 configured to acquire elasticity information such as strain or elasticity modulus from displacement information measured by displacement measuring unit 22;

elastic image constructing unit 24 configured to construct a color elastic image from the strain or elasticity modulus calculated by elasticity information calculating unit 23;

color scan converter 25 configured to convert the output signals of elastic image constructing unit 24 to make them match the display of image display unit 20;

switching and adding unit 19 configured to overlap, juxtapose or switch a black and white tomographic image and a color elastic image; and

image display unit 20 configured to display the composite image.

Also, it comprises:

distance measuring unit 30 configured to measure the distance in the reference points being set using the displacement information calculated by displacement measuring unit 22, and to cause image display unit 20 to display the distance;

elasticity information extracting unit 31 configured to extract elasticity information in the set region of interest, and to cause image display unit 20 to display the extracted elasticity information; and

pressure measuring unit 26 configured to measure the pressure condition of the surface of ultrasonic probe 11.

Ultrasonic diagnostic apparatus 1 of the present invention will be described below in detail. Ultrasonic probe 11 is formed by arranging a plurality of transducers, and has the function to transmit/receive ultrasonic waves to/from object 10 via the transducers. Transmission unit 13 has the function to generate a transmission pulse for generating an ultrasonic wave by driving ultrasonic probe 11, and to set the convergent point of the transmitted ultrasonic wave at a certain depth. Also, reception unit 14 amplifies the reflected echo signal received by ultrasonic probe 11 by a predetermined gain so as to generate an RF signal, i.e. reception signal. Transmission/reception separating unit 12 has the switching function for switching circuits for transmitting an ultrasonic wave generated by transmission unit 13 and outputting the reflected echo from object 10 to reception unit 14. Ultrasonic transmission/reception controller 15 controls transmission unit 13 and reception unit 14.

Phasing and adding unit 16 inputs, phases and adds the RF signals amplified in reception unit 14 so as to generate RF frame data by forming ultrasonic beams with respect to one or more convergent points.

Tomographic image constructing unit 17 executes signal processing such as gain compensation, log compression, detection, edge enhancement or filtering by inputting the RF frame data from phasing and adding unit 16, so as to obtain tomographic image data. Also, black and white scan converter 18 is configured including an A/D converter for converting the tomographic image data from tomographic image constructing unit into digital signals, a frame memory for storing the converted plurality of tomographic image data in time series, and a controller. The black and white scan converter 18 obtains the tomographic image data of object 10 stored in the frame memory as one image, and reads out the obtained tomographic image data by TV synchronous.

RF frame data selecting unit 21 stores the plurality of RF frame data from phasing and adding unit 16, and selects a pair of, i.e. two sets of RF frame data from among a group of stored RF frame data.

RF frame data selecting unit 21 is formed by a frame memory and a frame selector unit. The frame memory stores/updates the RF frame data for a given length of time (for example, for two heartbeats) in time series. The frame selector unit selects two frames that are most suitable for generating an elastic image from among the stored RF frame data, and outputs them to displacement measuring unit 22 in the subsequent stage. For example, the RF frame data generated from phasing and adding unit 16 based on the time series, i.e. frame rate of the image is sequentially stored in the frame memory, the stored RF frame data (N) is selected in the frame selector unit as a first data, and one RF frame data (X) is selected by the frame selector unit from among RF frame data group (N-1, N-2, N-3 . . . , N-M) which is temporally stored in the past. Here, N, M and X are index numbers appended to the RF frame data, and are natural numbers.

Then displacement measuring unit 22 executes one-dimensional or 2-dimensional correlation process from a selected pair of RF frame data, i.e. RF frame data (N) and RF frame data (X), and obtains the displacement or a movement vector, i.e. one-dimensional or two-dimensional displacement distribution in relation to the direction and the size of displacement in the biological tissue corresponding to the respective points on the tomographic image. Here, the block matching method is used for detecting the moving vector. The block matching method divides an image into blocks formed by, for example, N×N pixels, focusing on the block within a region of interest, searches for the block most approximate to the focusing block from the previous frame, and executes predictive coding, i.e. the process for determining the sample value by the difference referring to the searched block.

Elasticity information calculating unit 23 calculates strain or elasticity modulus of the biological tissue corresponding to the respective points on a tomographic image from the measurement value such as moving vector outputted from displacement measuring unit 22 and pressure value outputted from pressure measuring unit 26, and generates elasticity frame data based on the calculated strain or the elasticity modulus.

At this time, the strain is calculated by performing special differentiation on the moving distance, for example, the displacement of the biological tissue. Also, elasticity modulus is calculated by dividing the variation of pressure by the variation of displacement. For example, when the displacement measured by displacement measuring unit 22 is set as L(X) and the pressure measured by pressure measuring unit 26 is set as P(X), since strain ΔS(X) can be calculated by performing special differentiation on L(X), the formula to obtain the strain will be ΔS(X)=ΔL(X)/(X). Also, Young's modulus YM(X) of elasticity modulus data can be calculated by YM=(ΔP(X))/ΔS(X). Since the elasticity modulus of the biological tissue equivalent to the respective points on the tomographic image can be acquired from this Young's modulus YM, two-dimensional elastic image data can be continuously obtained. The Young's modulus is the ratio between the simple tension stress added to a substance and the strain generated in parallel to the tension.

Elastic image construction unit 24 executes normalization process or averaging process on the calculated elasticity frame data so as to facilitate stable generation of the continuously calculated elastic images.

Color scan converter 25 has a function to append hue information on elasticity frame data from elastic image construction unit 24. That is, it converts elasticity data into light's three primary colors that are red (R), green (C) and blue (B). For example, the elasticity data having large strain is converted into red code, and the elasticity data having small strain is converted into blue code.

Switching and adding unit 19 comprises therein a frame memory, an image processing unit and an image selecting unit. The frame memory stores tomographic image data outputted from black and white scan converter 18 and elastic image data outputted from color scan converter 25. The image processing unit synthesizes the tomographic image data stored in the frame memory and the elastic image data by modifying their synthesis ratio. The luminance information and hue information of the respective pixels of the synthetic image consist of the addition of information from each of the black and white tomographic image and the color elastic image using the synthesis ratio.

Further, the image selecting unit selects the image to be displayed on image display unit 20 from among the tomographic image data and elastic image data in the frame memory and the synthetic image data (elastic image data+tomographic image data) in the image processing unit.

As an example of the image to be displayed on image display unit 20, the long axis view upon imaging a carotid artery is shown in FIG. 2. The left part on the same diagram shows tomographic image 40. Also, the right part thereof shows the synthetic image of elastic image 41 and tomographic image 40.

Here, the first embodiment of the present invention will be described referring to FIG. 3˜FIG. 6.

Distance measuring unit 30 sets the length L (the length between the arterial walls) of line segment 52 formed by the two movement reference point 50 and movement reference point 51 being set on a tomographic image or elastic image on image display unit using console 28 as initial length D0, and measures the length L of line segment 52 based on displacement D1, D2, D1′ and D2′ of movement reference point 50 and movement reference point 51 measured by displacement measuring unit 22.

In concrete terms, as shown in FIG. 3 and FIG. 4, movement reference point 50 and movement reference point 51 are set using console 28 on the arterial wall of a carotid artery displayed by tomographic image 40. Here, in order to capture the movement of the arterial wall due to pulsation of the carotid artery, movement reference point 50 and movement reference point 51 are set on the arterial wall of the carotid artery to be facing each other. That is, line segment 52 formed by movement reference point 50 and movement reference point 51 is the same as the diameter of the carotid artery.

Then distance measuring means 18 calculates the distance in movement reference point 50 and movement reference point 51 using the method such as above-described block matching method. Distance measuring means 18 focuses on a block within the region of interest centering on movement reference point 50 and movement reference point 51, searches for the block which is most approximated to the focused block from the previous tomographic image data or elastic image data, determines the sample value referring to the searched block using predictive coding, i.e. difference, and measures the distance of movement reference point 50 and movement reference point 51.

Then as shown in FIG. 4, distance measuring means 18 adds or subtracts the measured distances D1, D2, D1′ and D2′ with respect to initial length D0. When the carotid artery is expanded in an expansion period, i.e. movement reference point 50 is moved upward and movement reference point 51 is moved downward, distance measuring means 18 adds distance D1 and D2 to initial length D0 and calculates length L of line segment 52 as the following formula.

D=D0+D1+D2 {Formula 1}

Also, when the carotid artery is contracted in a contraction period, i.e. movement reference point 50 is moved downward and movement reference point 51 is moved upward, distance measuring means 18 subtracts distance D1′ and D2′ from initial length D0 and calculates length L of line segment 52 as the following formula.

D=D0−D1′−D2′ {Formula 2}

Distance measuring means 18 repeats these calculations, and continuously calculates the length L of line segment 52 for a given length of time (for example, for 2 heartbeats).

Controller 27 may be configured so that movement reference point 50 and movement reference point 51 are set automatically using luminance information or Doppler information of tomographic image 40. For example, controller 27 sets movement reference point 50 at a place which is a part of the region extending in the lateral direction having high luminance within the region of interest on elastic image 41, where there is no Doppler signal. Then controller 27 sets movement reference point 51 at the place downward from movement reference point 50 which has high luminance.

Also, controller 27 may be configured to set movement reference point 50 and movement reference point 51 along the compression direction of ultrasonic probe 11.

Also, it may be configured, for example, to set first ROI 55 and second ROI 56 to be adjacent to the tunica interna and tunica externa of a carotid artery via console 28. At this time, first ROI 55 and second ROI 56 are set in the vicinity of the place where movement reference point 51 is being set.

For example, first ROI 55 and second ROI 56 are set so that movement reference point 51 is set on the tangent line of first ROI 55 and second ROI 56.

Elasticity information extracting unit 31 extracts elasticity information of first ROI 55 and second ROI 56. Elasticity information extracting unit 31 performs adding and averaging on the strain in the respective coordinates of first ROI 55 and calculates the average strain in first ROI 55. Also, elasticity information extracting unit 31 performs adding and averaging on the strain in the respective coordinates of second ROI 56 and calculates the average strain in second ROI 56.

Also, elastic information extracting unit 31 calculates the strain ratio of first ROI 55 with respect to second ROI 56 based on the average strain value in first ROI 55 and the average strain value in second ROI 56. That is, it calculates the strain ratio of the tunica interna with respect to the tunica externa. Also, it calculates the average luminance value of first ROI 55 and second ROI 56 from the black and white tomographic image data constructed in tomographic image constructing unit 17. Upon calculating the strain ratio, however, first ROI 55 and second ROI 56 may be reversed.

The average luminance value, the average strain value and the strain ratio extracted by elasticity information extracting unit 31 are displayed on image display unit 20.

As shown in FIG. 5, time sequence graph 60 of length L in line segment 52 formed by movement reference point 50 and movement reference point 51 is displayed in the middle part of image display unit 20. By the display of time series graph 60 of length L in line segment 52, it is possible to recognize what time zones are in an expansion period or in an extraction period of the carotid artery. When the length of length L of line segment 52 formed by movement reference point 50 and movement reference point 51 is long, the diameter of the short-axis view of the carotid artery is large. Therefore, the carotid artery is in the expanded state. Also, when the length of length L of line segment 52 formed by movement reference point 50 and movement reference point 51 is short, the diameter of the short-axis view of the carotid artery is small. Therefore, the carotid artery is in the contracted state.

Time series graph 60, when the arterial wall is in normal condition, shows the waveform characterized in having precipitous rising edge and moderate trailing edge, resembling blood pressure waveform closely. Since the characteristic often varies depending on illness, there are cases that the condition of illness can be confirmed from the measured waveform.

Also, in the middle part of image display unit 20, time series graph 61 indicating the average strain value in first ROI 55, time series graph 62 indicating the average strain value in second ROI 56 and time series graph 63 indicating the strain ratio are displayed. In the same manner, the time series graphs of the average luminance value can be displayed on image display unit 20. Here, from the starting-point to the ending-point is shown for the portion of two heartbeats.

With respect to the displayed time series graph, time gauges are set via console 28. Length L of line segment 52 formed by movement reference point 50 and movement reference point 51 in X-time phase of time gauge 64 being set on tomographic image 40 is displayed by a numerical value in lower part 66 of image display unit 20. Also, the average luminance value, the average strain value and the strain ratio of first ROI 55 and second ROI 56 in time gauge 65 which is in the same phase (X-time phase) as time gauge 64 are displayed by numerical values in the lower part 66.

The time zone that a carotid artery expands is the time zone that pressure is added to an arterial wall. Therefore, an operator can refer to the average luminance value, the average strain value and the strain ratio of first ROI 55 and second ROI 56 while confirming whether the carotid artery is in the expansion time zone or in the contraction time zone, whereby making it possible to measure elastic information accurately.

The second embodiment will be described referring to FIG. 6. The difference from the first embodiment is that time gauges can be set manually.

In the case of setting time gauge 64 in manual mode, an operator slides time gauge 64 to the left or to the right via console 28. Then controller 27 causes time gauge 64 to be slid based on the input information. For example, the operator sets time gauge 64 in the time zone that the carotid artery expands and the compression is added properly.

Also, controller 27 slides time gauge 65 to make it be in the same time phase as time gauge 64 which has been slid, in time series graph 61 indicating the average strain value in first ROI 55, time series graph 62 indicating the average strain value in second ROI 56 and time series graph 63 indicating the strain ratio.

Elasticity information extracting unit 31 displays on the lower part 66 the average luminance value, the average strain value and the strain ratio of first ROI 55 and second ROI 56 corresponding to the time phase of time gauge 64 and time gauge 65 which have been slid.

While time gauge 64 is set by manual mode via console 28 above, time gauge 64 can be set also by automatic mode. In concrete terms, controller 27 performs differentiation on the waveform of time series graph 60 of length L of line segment 52 formed by movement reference point 50 and movement reference point 51. When the slope (differential value) rises, it is in the time zone of an expansion period and the compression is applied properly. Controller 27 slides time gauge 64 so that the time gauge 64 is set in the time zone where the slope (differential value) rises. Controller 27 slides time gauge 65 to left and right to make it to be set in the same time phase as time gauge 64. In this manner, it is possible to set time gauge 64 and time gauge 65 in the time zone wherein compression is applied properly.

As shown in FIG. 6, the waveforms in time series graph 60 consist of a time zone where the slope (differential value) rises upward from left to right and a time zone where the slope (differential value) descends downward from left to right, and these time zones exist alternately. Also, alternate time zones wherein the slope (differential value) rises and descends continue to exist for a given length of time. Since the timing when the slope (differential value) varies from upward to downward is somewhat influenced by a factor such as noise, controller 27 slides, for example, time gauge 64 to be set near the center of the time zone where the slope (differential value) rises, as shown in time gauge 64 of FIG. 5. Then controller 27 slides time gauge 65 to the left or right to be in the same time phase as time gauge 64.

Also, a plurality of time gauges may also be set. As shown in FIG. 6, time gauge 70 in addition to time gauge 64 is set via console 28. Length L of line segment 52, in time phase Y of time gauge 70, formed by movement reference point 50 and movement reference point 51 is displayed by a numerical value in lower part 67 of image display unit 20. Also, the average luminance value, the average strain value and the strain ratio in first ROI 55 and second ROI 56 of time gauge 71 which is in the same time phase as time gauge 65 (time phase Y) are displayed in numerical values in lower part 67, whereby making it possible to measure the elasticity information accurately.

For example, by setting time gauge 64 (time gauge 65) and time gauge 70 (time gauge 71) when the carotid artery is expanded by the above-mentioned manual mode or automatic mode, it is possible to perform comparison in relation to the length L of line segment 52, the average luminance value, the average strain value and the strain ratio of first ROI 55 and second ROI 56 in time phase X and time phase Y.

For example, length L of line segment 52, the average luminance value, the average strain value and the strain ratio in time phase X and time phase Y being set in the expansion period of the carotid artery are the same, it means that the values in the both phases are reliable. While two time gauges (two time phases) are set in the example above, more than two time gauges (time phases) may be set.

Next, the third embodiment will be described referring to FIG. 7. The difference from the first and second embodiments is that the average luminance value, the average strain value and the strain ratio of length L of line segment 52, first ROI 55 and second ROI 56 are measured in a predetermined time zone.

Controller 27 sets time gauge 81 and time gauge 83 at the timing when the slope of a waveform (differential value) of time series graph 60 changes from downward to upward (local minimum value). Also, controller 27 sets time gauge 82 and time gauge 84 at the timing when the slope of a waveform (differential value) of time series graph 60 changes from upward to downward (local maximum value). Time gauge 81˜time gauge 84 are respectively set in the time series order.

Controller 27 sets time gauge 85 which is in the same time phase as time gauge 81 in time series graph 61 of the average strain value in first ROI 55, time series graph 62 of the average strain graph in second ROI 56 and time series graph 63 of the strain ratio. In the same manner, time gauge 86 which is in the same time phase as time gauge 82, time gauge 87 which is in the same time phase as time gauge 83 and time gauge 88 which is in the same time phase as time gauge 84 are set by controller 27.

Here, the time zone sandwiched between time gauge 81 and time gauge 82 is set as time zone A, the time zone sandwiched between time gauge 82 and time gauge 83 is set as time zone B, the time zone sandwiched between time gauge 83 and time gauge 84 is set as time zone C, and the time zone in the time phase after time gauge 84 is set as time zone D. The carotid artery expands in time zone A and time zone C, and the carotid artery contracts in time zone B and time zone D.

Elasticity information extracting unit 31 displays the measurement value calculated by dividing the cumulative value of length L of line segment 52, the average luminance value, the average strain value and the strain ratio of first ROI 55 and second ROI 56 measured in time zone A when the carotid artery is expanded by the number of measurement points (time average of the respective measurement values measured in time zone A may also be used), on lower part 90 respectively. In the same manner, elasticity information extracting unit 31 displays the respective measured values calculated in time zone B on lower part 91, displays the respective measured values calculated in time zone C on lower part 92, and displays the respective measured values calculated in time zone D on lower part 93.

Elasticity information extracting unit 31 is capable of measuring elasticity information accurately by displaying the measurement values of time zone A and time zone C on lower part 90 and referring to the average luminance value, the average strain value and the strain ratio of first ROI 55 and second ROI 56 in the time zone of the expansion period and the contraction period of the carotid artery.

While the measured value in time zone A which is in the expansion period of the carotid artery in the first cycle is described above, elasticity information extracting unit 31 may display plural cycles (time zone A and time zone C) of measured values. For example, elasticity information extracting unit 31 is capable of displaying the measured value calculated by dividing the accumulative value of length L of line segment 52, the average luminance value, the average strain value and the strain ratio of first ROI 55 and second ROI 56 in time zone A and time zone C when the carotid artery is expanded by the measurement score (time average of the respective measurements measured in time zone A and time zone C may also be used), on lower part 90 respectively.

Next, the fourth embodiment will be described referring to FIG. 5 and FIG. 7. The difference from the first embodiment˜the third embodiment is the change of display patterns by the time zone of the expansion period of the carotid artery and the time zone of the contraction period.

As shown in the third embodiment, time zone A˜time zone D are set first. The image selecting unit of switching and adding unit 19 selects the image to be displayed on image display unit 20 from among the tomographic image data and elastic image data in the frame memory and the synthetic image data in the image processing unit, based on the time zone set by controller 27. In concrete terms, the image selecting unit of switching and adding unit 19 selects the elastic image data or synthetic image data (elastic image data+tomographic image data) in time zone A or time zone C. Also, the image selecting unit of switching and adding unit 19 selects the tomogrpahic image data in time zone B or time zone D.

Time zone A and time zone C are suitable for displaying elastic image 41, since it is the time zone when the carotid artery is expanded and compression is added to the arterial wall. Therefore, elastic image 41 is displayed in time zone A and time zone C. Also, time zone B and time zone D are not suitable for displaying elastic image 41, since they are the time zones when the carotid artery is contracted including the timing that compression which is not appropriate for generating the elastic image is added. Therefore, elastic image 41 is not displayed in time zone B and time zone D.

As mentioned above, since elastic image 41 is displayed only in the time zones when the carotid artery is expanded, only elastic images of which the compression is applied appropriately can be displayed, whereby making it possible to achieve accurate display of elastic images.

Next, the fifth embodiment will be described referring to FIG. 8. The difference from the first embodiment˜the fourth embodiment is the change of display patterns of time series graphs 60˜63 by the time zone for expansion period and the time zone for contraction period of the carotid arteries.

As shown in the third embodiment and the fourth embodiment, time zone A˜time zone D are set first. Distance measuring unit 30 changes the display pattern of time series graph 60 of length L of line segment 52 formed by movement reference point 50 and movement reference point 51, based on the time zone set by controller 27. In concrete terms, as shown in FIG. 8(a), distance measuring unit 30 causes image display unit 20 to display time series graph 60 in time zone A or time zone C. Also, distance measuring unit 30 causes image display unit 20 not to display time series graph 60 in time zone B or time zone D. As shown in FIG. 8(b), distance measuring unit 30 causes image display unit 20 to display time series graph 60 by a solid line in time zone A or time zone C. Also, distance measuring unit 30 causes image display unit 20 to display time series graph 60 by a broken line or a dotted line in time zone B or time zone D. It may be configured so that distance measuring unit 30 causes image display unit 20 to display time series graph 60 in time zone A or time zone C in blue and to display time series graph 60 in time zone B or time zone D in red.

As shown in FIG. 8(a), elastic image extracting unit 31 changes the display patterns of time series graph 61 of the average strain value in first ROI 55, time series graph 62 of the average strain value in second ROI 56 and time series graph 63 of the strain ratio, based on the time zone set by controller 27. In concrete terms, elasticity information extracting unit 31 causes image display unit 20 to display time series graph 61˜time series graph 63 in time zone A or time zone C. Also, elasticity information extracting unit 31 causes image display unit 20 not to display time series graph 61˜time series graph 63 in time zone B or time zone D. As shown in FIG. 8(b), elasticity information extracting unit 31 causes image display unit 20 to display time series graph 61˜time series graph 63 in time zone A or time zone C by a solid line. Also, elasticity information extracting unit 31 causes image display unit 20 to display time series graph 61˜time series graph 63 in time zone B or time zone D by a broken line or a dotted line. It may be configured so that elasticity information extracting unit 31 causes image display unit 20 to display time series graph 61˜time series graph 63 in time zone A or time zone C in blue and to display time series graph 61˜time series graph 63 in time zone B or time zone D in red.

In this manner time zone A and time zone C are suitable for displaying time series graph 60˜time series graph 63, since it is the time zone when the carotid artery is expanded and the compression is added to the arterial wall. Therefore, time series graph 60˜time series graph 63 are displayed in time zone A and time zone C distinctively. Also, time zone B and time zone D are the time zones when the carotid artery is contracted and the blood pressure level gradually declines. When getting closer to the end of the contraction period, the time that the significant difference is not generated in the calculated strain value or strain ratio is also included. Time zone B and time zone D are not suitable for displaying time series graph 60˜time series graph 63, since they are the time zones when the compression is not applied appropriately. Therefore, time series graph 61˜time series graph 63 are displayed in time zone B and time zone D inconspicuously.

Consequently, it is possible to achieve accurate measurement of elastic information.

Next, the sixth embodiment will be described referring to FIG. 6 and FIG. 9. The difference from the first embodiment˜the fifth embodiment is that the setting positions of first ROI 55 and second ROI 56 are modified in accordance with the pressure. For example, the setting positions of first ROI 55 and second ROI 56 are modified based on length L of line segment 52 formed by movement reference point 55 and movement reference point 56.

As shown in FIG. 6, along with pulsation of the carotid artery, movement reference point 50 moves upward and movement reference point 51 moves downward. Movement reference point 50 and movement reference point 51 move upward and downward about the same distance from each other. In other words, the half of the variation of length L of line segment 52 measured in distance measuring unit 30 is the distance that movement reference point 50 and movement reference point 51 moved. In the same manner, as shown in FIG. 9, controller 27 moves first ROT 55 and second ROI 56 along with movement reference point 51.

In the case that length L of line segment 52 is long, controller 27 moves first ROI 55 and second ROI 56 downward for the half portion of variation of length L of line segment 52 based on the variation of movement reference point 50 and movement reference point 51. In the case that length L of line segment 52 is short, controller 27 moves first ROI 55 and second ROI 56 upward for the half portion of variation of length L of line segment 52 based on the variation of movement reference point 50 and movement reference point 51.

In this manner, moving first ROI 55 and second ROI 56 along with the pulsation of the carotid artery makes it possible to set first ROI 55 on the tunica interna of the carotid artery and second ROI 56 on the tunica externa of the carotid artery appropriately. Therefore, the average luminance value, the average strain value and the strain ratio of first ROI 55 and second ROI 56 can be accurately measured, whereby making it possible to achieve accurate measurement of the elasticity information.

While first ROI 55 and second ROI 56 are moved upward and downward by controller 27 for the half portion of the variation of length L of line segment 52 in the above description, there are cases that movement reference point 50 and movement reference point 51 do not move upward and downward for the same portion as each other due to influence of the factor such as an adjacent bone. In such cases, it is possible to set what percentage of the variation of length L of first ROI 55 and second ROI 56 should be moved upward and forward in controller 27.

Also, controller 27 may be set so that a reference point is set on each of first ROI 55 and second ROI 56 via console 28, and first ROI and second ROI are made to follow the set reference points by applying the method such as the above-described block matching method.

Next, operation procedure of the present invention will be described referring to FIG. 10.

(Step 1): Ultrasonic waves are transmitted/received to/from a carotid artery which is the measuring target, and tomographic image 40 and elastic image 41 are displayed on image display unit 20 based on the RF frame data.
(Step 2): The RF frame data for a given length of time (for example, for two pulsations) is stored and updated in a frame memory. Then tomographic image 40 and elastic image 41 are performed with the freezing process using a freezing button of console 28. The update of RF frame data, along with the freezing, is stopped and the RF frame data for a given length of time (for example, for two pulsations) is stored.
(Step 3): Movement reference point 50 and movement reference point 51 are set via console 28 in order to measure the diameter of the arterial wall which varies in compliance with the pulsation.
(Step 4): First ROI 55 and second ROI 56 are set via console 28 to be adjacent to the tunica interna and the tunica externa of first ROI 55 and second ROI 56.
(Step 5): The RF frame data stored in RF frame data selecting unit 21 is read in by displacement measuring unit 22, and displacement distribution of the arterial wall between the frames that varies in compliance with the pulsations is calculated. Distance measuring unit 30 calculates the length of line segment 52 of movement reference point 50 and movement reference point 51 being set on the arterial wall from the calculated displacement distribution data. Also, elasticity information calculating unit 23 calculates the strain distribution data from the displacement distribution data. Elasticity information extracting unit 31 calculates the average strain value and the strain ratio of first ROI 55 and second ROI 56 from the calculated strain distribution data. Elasticity information extracting unit 31 calculates the average luminance value of first ROI 55 and second ROI 56 from the black and white tomographic data outputted from tomographic image constructing unit 17.
(Step 6): The variation of the length of line segment 52 based on movement reference point 50 and movement reference point 51, the average luminance value, the average strain value and the strain ratio calculated from first ROT 55 and second ROI 56 are displayed on image display unit 20 by numeric values and time series graphs. The length of line segment 52 calculated based on movement reference point 50 and movement reference point 56 indicates the distance between the arterial walls.

Also, time series graph 61 of the average strain value in first ROI 55, time series graph 62 of the average strain value of second ROI 56 and time series graph 63 of the strain ratio are displayed on image display unit 20. Since the form or amplitude of waveforms vary depending on the progress of disease, it is effective to capture the variation of disease with passage of time, not only the elasticity information by numeric values at the present time.

By above-described steps, distance between the arterial walls, the average strain value or the strain ratio can be learned by corresponding to each piece of information while confirming the tomographic image or elastic image, whereby making it possible to achieve accurate measurement of elasticity information.

(Step 7): Also, time gauge 64 and time gauge 65 can be set on the displayed time-series graphs via console 28. Time gauge 64 and time gauge 65 can be set in a segment from a starting-point to an ending-point.
(Step 8): Time gauge 64 is displayed on time series graph 60 showing the distance between the arterial walls. Time gauge 64 is displayed on time series graph 61 of the average strain value in first ROI 55, time series graph 62 of the average strain value in second ROI 56 and time series graph 63 of the strain ratio. The set time gauge 64 and time gauge 65 are for indicating the same time phases, making it possible to display the respective measurement information in the same timing.
(Step 9): A plurality of time gauges 81˜84 and time gauges 85˜88 are set with respect to the displayed time series graphs via console 28. Time gauge 81 and time gauge 83 are set in the timing that the slope of the waveform (differential value) of time series graph 60 varies from upward to downward (minimum value). Also, controller 27 sets time gauge 82 and time gauge 84 in the timing that the slope of the waveform (differential value) of time series graph 60 varies from upward to downward (maximum value). In this manner, the time zone can be divided into the contraction period and the expansion period. By dividing the timings, the accurate measurement of elasticity information can be achieved in the divided contraction period and the expansion period.
(Step 10): The length of the line segment, the average luminance value, the average strain value and the strain ratio in the time zones A˜D segmented by time gauges 81˜84 are displayed on image display unit 20 by numerical values. In this manner, when the timings are divided into the contraction period and the expansion period of the carotid artery, the average values in the respective time zones are displayed on image display unit 20.

Also, by matching time gauges 81˜84 to the starting point˜ending point, the length of the line segment, the average luminance value, the average strain value and the strain ratio during a given 1 pulsation can be displayed. In the case that the time gauges are moved from time phase X to time phase Y on console 28, the respective numerical values are updated to the value of time phase Y in accordance with the movement.

1. An ultrasonic diagnostic apparatus comprising:
an ultrasonic probe;
tomographic image constructing means configured to generate a tomographic image based on the RF frame data obtained from the ultrasonic probe, of the cross-sectional region of an object to be examined;
elasticity information calculating means configured to obtain strain and/or elasticity modulus of a tissue in the cross-section region based on the RF frame data;
elastic image constructing means configured to construct an elastic image of the cross-sectional region based on the strain and/or elasticity modulus acquired in the elasticity information calculating means; and
display means configured to display the tomographic image and/or the elastic image,
characterized in further comprising:
setting means configured to set a first movement reference point and a second movement reference point on the tomographic image and/or the elastic image; and
measuring means configured to make the first movement reference point and the second movement reference point follow the pulsation of the object, and to measure the length of a line segment formed by the first movement reference point and the second movement reference point,
and causes the display means to display the length of the line segment and the elasticity information based on the strain and/or the elasticity modulus.

2. The ultrasonic diagnostic apparatus according to claim 1, wherein the setting means sets a region of interest in the vicinity of the first movement reference point and the second movement reference point, so that the elasticity information of the region of interest is displayed on the display means.

3. The ultrasonic diagnostic apparatus according to claim 2, wherein the elastic information is the average strain value within the region of interest.

4. The ultrasonic diagnostic apparatus according to claim 2, wherein the elasticity information is the strain ratio between the two regions of interest.

5. The ultrasonic diagnostic apparatus according to claim 1, wherein the display means displays a time series graph based on the length of a line segment and/or the elasticity information.

6. The ultrasonic diagnostic apparatus according to claim 4, wherein the display means displays a time gauge showing a time phase on the time series graph.

7. The ultrasonic diagnostic apparatus according to claim 1, wherein the measuring means measures the length of a line segment or the elasticity information in a predetermined time zone.

8. The ultrasonic diagnostic apparatus according to claim 1, wherein the display means changes display pattern of the elasticity information based on variation information of the length of a line segment.

9. The ultrasonic diagnostic apparatus according to claim 5, wherein the display means changes display pattern of the time series graph based on variation information of a line segment.

10. The ultrasonic diagnostic apparatus according to claim 2, wherein the setting means changes the setting position of the region of interest in accordance with pressure.
